(19) United States
(12) Patent Application Publication
(10) Pub. No.: US 2010/0217270 A1
(43) Pub. Date: Aug. 26, 2010

(54) Integrated Production of Patient-Specific Implants and Instrumentation

(75) Inventors: Martin J. Polinski, Wrentham, MA (US); Paula M. Berg, Bolton, MA (US)

Correspondence Address: Sunstein Kann Murphy & Timbers LLP, 125 SUMMER STREET, BOSTON, MA, 02110-1618, US

(73) Assignee: CONFORMIS, INC., Burlington, MA (US)

(21) Appl. No.: 12/712,827

(22) Filed: Feb. 25, 2010

Related U.S. Application Data
(60) Provisional application No. 61/155,346, filed on Feb. 25, 2009.

(51) Int. Cl. A61B 17/56; G06F 17/50

(52) U.S. Cl. 606/87; 700/103; 700/98; 700/119

(57) ABSTRACT

Disclosed herein are devices, systems and methods for the automated design and manufacture of patient-specific/patient-matched orthopedic implants. While the embodiments described herein specifically pertain to unicompartmental resurfacing implants for the knee, the principles described are applicable to other types of knee implants (including, without limitation, other resurfacing implants and joint replacement implants) as well as implants for other joints and other patient-specific orthopedic applications.

### CROSS-REFERENCE TO RELATED APPLICATIONS

This application claims priority to U.S. Provisional Application No. 61/155,346, filed Feb. 25, 2009, which is incorporated herein by reference in its entirety.

This application is related to U.S. Ser. No. 11/671,745, filed Feb. 6, 2007, entitled “Patient Selectable Joint Arthroplasty Devices and Surgical Tools”, which is hereby incorporated by reference in its entirety.

### BACKGROUND OF THE INVENTION

1. Field of the Invention

The embodiments described herein relate to systems for designing and manufacturing patient-specific orthopedic devices, such as implants and instrumentation, based on data, such as imaging data, representing an existing joint. In particular, integrated devices and systems used to manufacture such devices are described.

2. Description of the Related Art

Traditional orthopedic implants, designed for mass manufacture, have a limited range of sizes and shapes. Fitting these implants to the patient requires extensive bone resection and shaving to alter the joint to fit the shape of the implant. The surgical technique for a total knee replacement, for example, is invasive and requires sacrificing healthy bone stock in addition to the diseased area. In addition, the hospital must carry a large range of surgical instrumentation, e.g., cutting blocks, spacers, and instruments that need to be stored and sterilized after each procedure.

Despite the challenges, total knee replacement is one of the most common surgical procedures. It is estimated that 19 million Americans seek medical attention for knee pain according to the American Association of Orthopedic Surgeons, and, of those, about 500,000 undergo knee replacement surgery.

Personalized medicine is one of the fastest growing trends in the healthcare industry. While this trend has mainly been seen in the drug sector, medical device manufacturers have also recognized the benefits of individualizing their products to meet the needs of different patient groups. The orthopedic implant manufacturers have recently launched implants optimized for different genders or geographies. However, these are not truly personalized, patient-specific or patient-matched approaches. Technological advances now allow for the design and manufacture of implants and associated instrumentation optimized for a specific individual. Such implants fall on a spectrum from, e.g., implants that are based on one or two aspects or dimensions of a patient's anatomy (such as a width of a bone, a location of a defect, etc.) to implants that are designed to conform entirely to that patient's anatomy and/or to replicate the patient's kinematics.

One example of such patient-specific or patient-matched technology is the ConforMIS iFit® technology used in the iUni® (unicompartmental knee resurfacing implant) and iDuo® (dual compartmental knee resurfacing implant). This technology converts Computed Axial Tomography (“CT”) or Magnetic Resonance Imaging (“MRI”) scans into individualized, minimally invasive articular replacement systems capable of establishing normal articular shape and function in patients with osteoarthritis. By starting with imaging data, the approach results in implants that conform to bone or cartilage, and reduce the need for invasive tissue resection. The implant is made to fit the patient rather than the reverse. By designing devices that conform to portions of the patient's anatomy, the implants allow the surgeon to resurface rather than replace the joint, providing for far more tissue preservation, a reduction in surgical trauma, and a simplified technique.

The image-to-implant process begins with the patient having a CT or MRI scan, which can be done on commonly available machines, using a standardized protocol that ensures the data needed to design the implant is captured properly. The image data is then combined with computer-aided design (CAD) methods to generate a patient-specific model of the knee from which a patient-specific implant and/or patient-specific instrumentation can be designed and manufactured. The electronic design file created during this process is used to fabricate the patient-specific implant and custom instrumentation, which is a process that takes approximately four to six weeks.

The development and manufacture time associated with all types of patient specific devices could be made more efficient by employing various forms of Direct Digital Manufacture (“DDM”) and/or Rapid Prototyping systems which allow for the patient specific implants, instrumentation and other devices to be more effectively produced and packaged. Such systems could result in, among other advantages, faster and less costly production, as well as a reduction in the cost of instrumentation, which is a significant capital expenditure associated with traditional orthopedic joint implants.

### SUMMARY OF THE INVENTION

Some embodiments described herein include a device for easily and efficiently manufacturing patient-specific instrumentation and implants. In one embodiment, a set of instrumentation used in an orthopedic surgical procedure is manufactured as one single piece in which all of the individual instruments are interconnected in a frame. The individual pieces can be removed from the frame as needed, including, without limitation, during the course of the surgical procedure. This allows the instruments to be manufactured efficiently and relatively less expensively. Furthermore, the instruments can be easily managed within the supply chain of the manufacturer, the doctor, and the hospital. For example, the instrumentation can be shipped to the hospital in a sterile condition when needed, and the instruments can be disposed of following surgery such that the hospital does not need to sterilize the instruments or maintain an expensive inventory of instruments before or after the surgery. Additionally, the instruments can be organized and controlled for the surgeon during the surgery.

In some embodiments, the instruments can be arranged in the order they will be used during the surgery to make it easier for the physician to identify and retrieve the instruments during surgery.

In other embodiments, patient-specific or patient-matched instruments are manufactured by, for example, a rapid prototyping process, while other non-patient-specific instruments may be manufactured by other processes, such as injection molding, or other process more suitable to mass production of identical parts.

### BRIEF DESCRIPTION OF THE DRAWINGS

The foregoing features will be more readily understood by reference to the following detailed description, taken with reference to the accompanying drawings, in which:

FIG. 1 is a top perspective view of an integrated patient-specific instrument set, in accordance with an embodiment of the invention.

FIG. 2 is a side perspective view of a representation of instruments in a CAD file, in accordance with an embodiment of the invention.

FIG. 3 is a side perspective view of a set of units in a manufacturing chamber, in accordance with an embodiment of the invention.

FIG. 4 is a flowchart of an Exemplary Manufacturing Process, in accordance with an embodiment of the invention.

FIG. 5 is a top perspective view of another embodiment of an integrated patient-specific instrument set, in accordance with an embodiment of the invention.

FIG. 6 is a side perspective view of the integrated patient-specific instrument set of FIG. 3.

### DETAILED DESCRIPTION

Patient-specific or patient-matched instruments can be manufactured as a single integrated unit or, alternatively, as several integrated units. Embodiments employing such integrated units or combinations of units may allow for more efficient manufacturing, control of inventory and/or more organization prior to and during surgery. Other embodiments may have additional or different advantages. Preferably, such integrated units are constructed using a rapid prototyping (“RPT”) process, Direct Digital Manufacturing (“DDM”) or other process suitable for manufacturing unique individual units or other devices that would be manufactured either as a one-off or low volume item.

Rapid prototyping is the automatic construction of physical objects using solid freeform fabrication. The first techniques for rapid prototyping became available in the late 1980s and were used to produce models and prototype parts. Today, they are used for a much wider range of applications and are even used to manufacture production quality parts in relatively small numbers. Some sculptors use the technology to produce complex shapes for fine arts exhibitions.

Rapid prototyping takes virtual designs from computer aided design (CAD) or animation modeling software, transforms them into thin, virtual, horizontal cross-sections and then creates each cross-section in physical space, one after the next until the model is finished. The virtual model and the physical model correspond almost identically, but may vary depending on the resolution used in the RPT process.

With additive fabrication, the machine reads in data from a CAD drawing and lays down successive layers of liquid, powder, or sheet material, and in this way builds up the model from a series of cross sections. These layers, which correspond to the virtual cross section from the CAD model, are joined together or fused automatically to create the final shape. The primary advantage to additive fabrication is its ability to create almost any shape or geometric feature.

The standard data interface between CAD software and the machines is the STL file format. An STL file approximates the shape of a part or assembly using triangular facets. Smaller facets produce a higher quality surface.

The word “rapid” is relative: construction of a model with contemporary methods can take from several hours to several days, depending on the method used and the size and complexity of the model. Additive systems for rapid prototyping can typically produce models in a few hours, although it can vary widely depending on the type of machine being used and the size and number of models being produced simultaneously.

Some solid freeform fabrication techniques use two materials in the course of constructing parts. The first material is the part material and the second is the support material (to support overhanging features during construction). The support material is later removed by heat or dissolved away with a solvent or water.

Traditional injection molding can be less expensive for manufacturing polymer products in high quantities, but additive fabrication can be faster and less expensive when producing relatively small quantities of parts. Recent advances in RPT processes now make the process cost effective for post-prototype manufacturing.

A large number of competing technologies are available in the marketplace. As all are additive technologies, their main differences are found in the way layers are built to create parts. Some melt or soften material to produce layers, while others use layers of liquid materials that are cured. In the case of lamination systems, thin layers are cut to shape and joined together. Among the various RPT technologies are selective laser sintering (SLS), direct metal laser sintering (DMLS), fused deposition modeling (FDM), selective laser melting (SLM), stereolithography (SLA), laminated object manufacturing (LOM), electron beam melting (EBM), Laser Engineered Net Shaping™ (LENS®), laser cladding, and 3D printing (3DP). (LENS® and Laser Enginered Net Shaping™ are registered trademarks of Sandia National Labs. and Sandia Corp.)

Rapid prototyping and direct digital manufacturing can be used to create various types of implants, including, without limitation, metal implants, as well as the associated instrumentation, including, without limitation, instrumentation made of synthetic materials. By using RPT and DDM processes to manufacture such devices, they can be cost-effectively produced. However, related implants and instruments can be manufactured even more cost-effectively by integrating the production into one or several associated units. This has the advantage not only of being a cost-efficient way to manufacture each individual piece, but the overall cost can be further reduced by manufacturing the devices in associated sets of devices. This may decrease cost directly by decreasing the actual cost of manufacture compared to separately manufacturing individual devices and components. Additionally, indirect costs may also be reduced, for example, by allowing streamlined management of the supply and inventory chains as well as quality control, because individual pieces no longer need to be tracked. Instead, only one or a few units containing the individual components in an integrated set can be tracked more easily with less chance for misplacing or confusing components with components associated with other patients. Additionally, the units can be marked with a serial number or other identifier that corresponds both with the patient, and the patient's implant to ensure that the proper implant and instrumentation is used on the patient during surgery.

A wide range of embodiments are possible, but the following two embodiments, a single integrated unit containing all patient-specific instrumentation plus some additional standard instrumentation, are described as exemplary embodiments. Many other embodiments, including, without limitation, many other configurations, arrangements, and combinations of units, are possible, however.

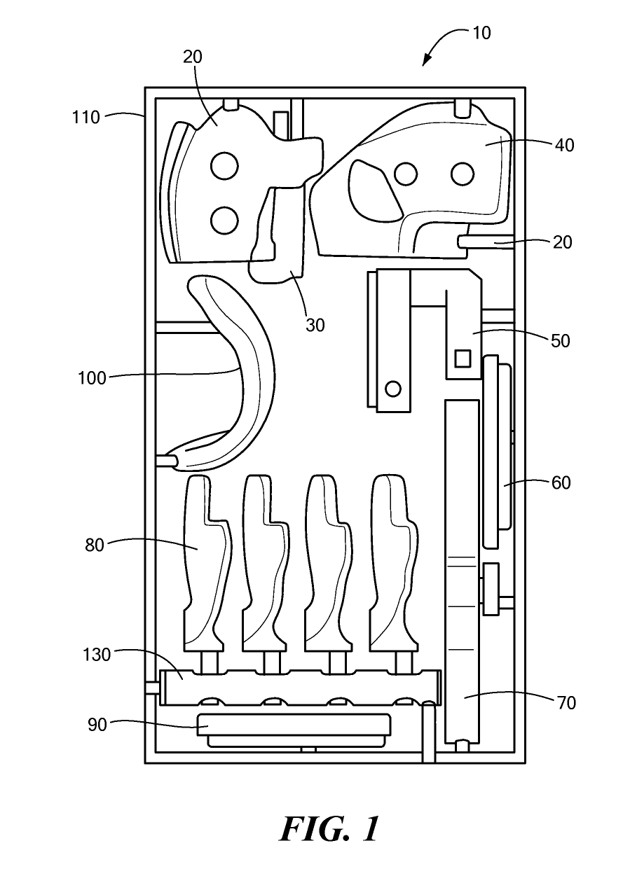

Referring to FIG. 1, a set of instruments is manufactured as an integrated unit 10. Unit 10 includes the patient-specific instruments to be used in a knee resurfacing operation in which a ConforMIS iUni unicompartmental knee resurfacing implant is to be implanted in a patient. The unit 10 includes a femoral guide 20, an “L”-guide 30, a femoral gauge 40, a tibial guide 50, a 7 mm tibial sample insert 60, a tibial template 70, a set of four balancing chips 80 (each being a different graduated size), a 9 mm tibial sample insert 90, and a femoral trial implant 100. Each of the components is included within a frame 110, and all of the components are manufactured as a single integrated piece. The components are connected by a set of pegs 120 and connectors 130 that attach each component to the frame 110, making the end product a single integrated unit that can be disassembled into the individual instrumentation components at the time of surgery.

The unit 10 is made of a nylon (PA2200 polyamid) and is manufactured by a selective laser sintering process. Although many manufacturing options exist, one such option is the EOS Formiga P100. Many other materials and processes could be used. Preferably, the material is autoclavable, or otherwise capable of being sterilized for surgical procedures.

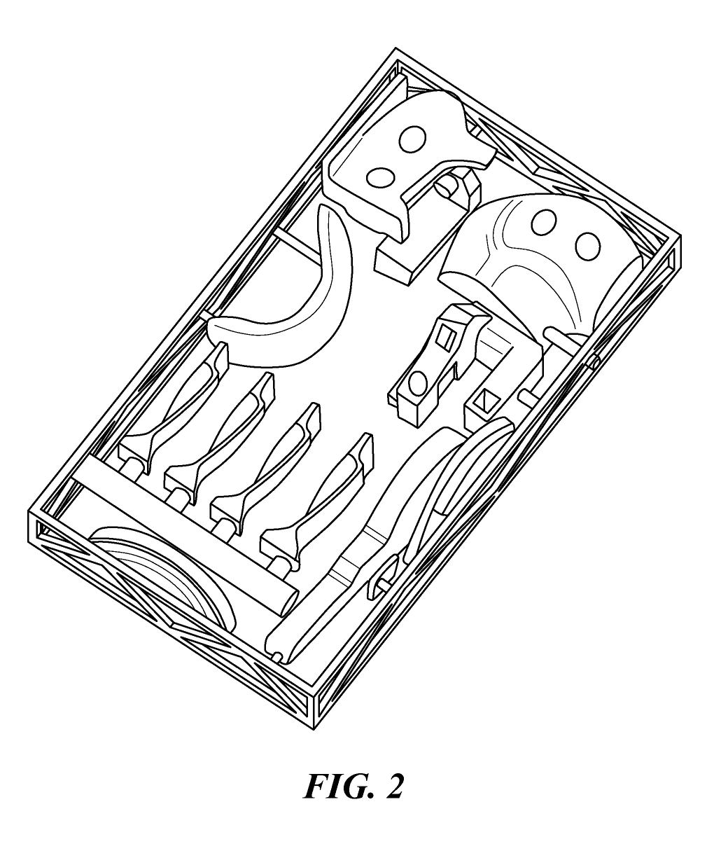

The instruments are designed using CAD technology and a data file is created that contains the specifications of the instruments to be produced. Referring to FIG. 2, a graphical representation of the data file used to produce instruments 10 is shown. In one embodiment, an assembly file is created that refers to the machining files that specify each of the individual components. The assembly file is used in conjunction with a higher level layer file to construct the final arrangement of components within the frame, and the positions of the pegs and other connectors are selected and provided in the assembly file. The assembly file is then converted to an STL file which is then used by a rapid prototyping machine to fabricate the instrumentation.

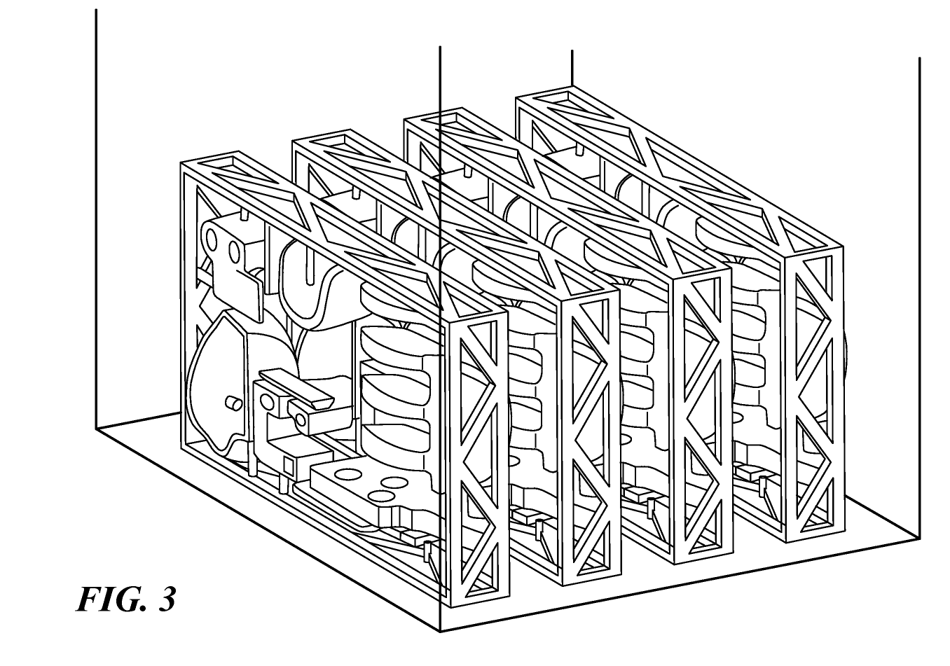

Several units can be manufactured during one run of the rapid prototyping machine, as shown in FIG. 3.

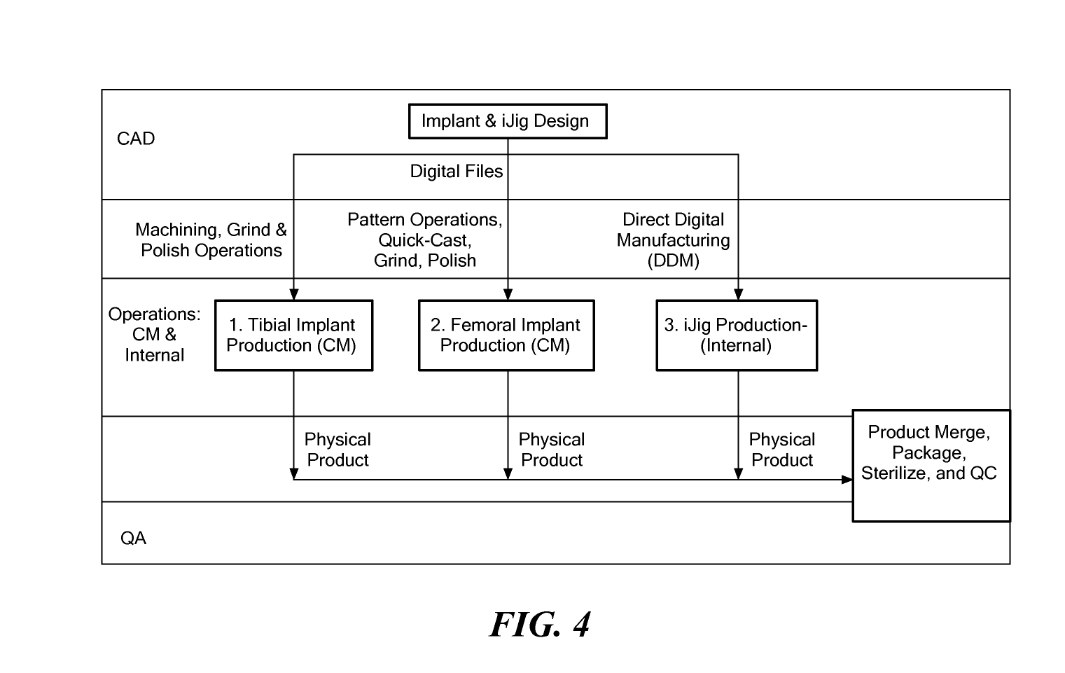

An exemplary manufacturing process that incorporates the instrumentation of Unit 10 is shown in FIG. 4. In this embodiment, the patient specific surfaces associated with the implant and the instrumentation are derived from a scan, e.g. a CT scan, MRI scan or other scan, of the patient's knee joint. The data are used to generate a CAD file of the implant, which includes in at least a portion, data from the derived joint surface, e.g. at the undersurface of the implant. From this CAD file of the implant, a jig system which includes a surface that is substantially a negative of the derived articular surface, is created. From here the process referred to earlier is followed by creating an assembly file, converting it to an STL file and sending it to a rapid prototyping machine.

In an alternative embodiment, the implant itself is included as part of the DDM manufacturing process and incorporated with or merged into the frame, a tray, or other similar structure. In such an embodiment, the CAD file for the implant is used to print either the entire implant or portions of the implant, such as the femoral component, using the Direct Digital Manufacturing process and a pattern of the implant is generated.

Many other combinations of components are possible. For example, standard parts that do not vary from patient-to-patient may be included, such as a set of standard tibial spacers (e.g., 9 and 11 mm spacers). Alternatively, all standard-sized parts can be manufactured together as one or more integrated units using another manufacturing process that may be more cost effective for such parts produced in bulk, such as injection molding.

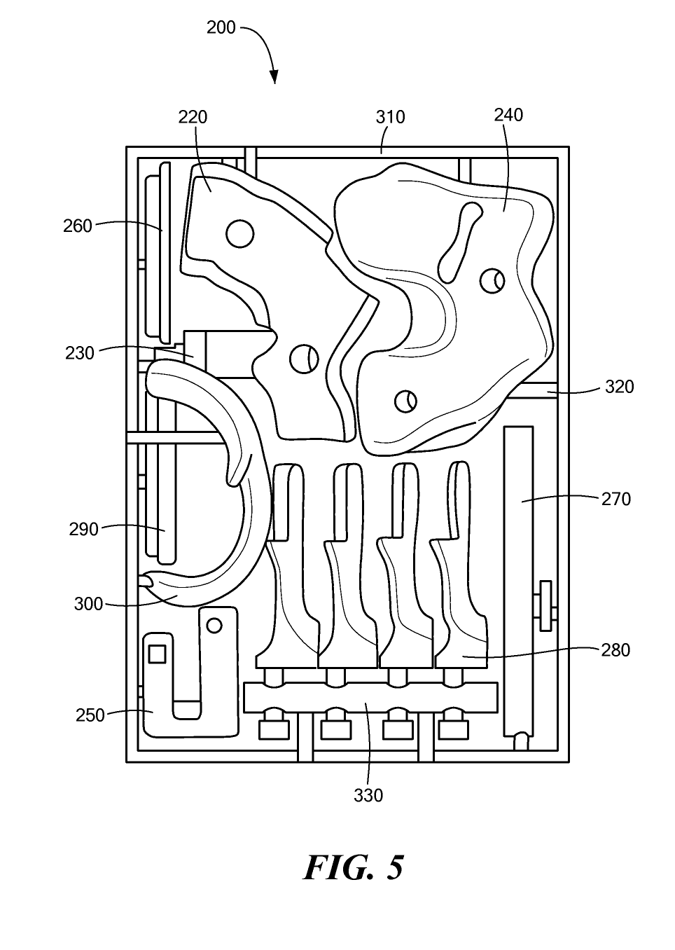

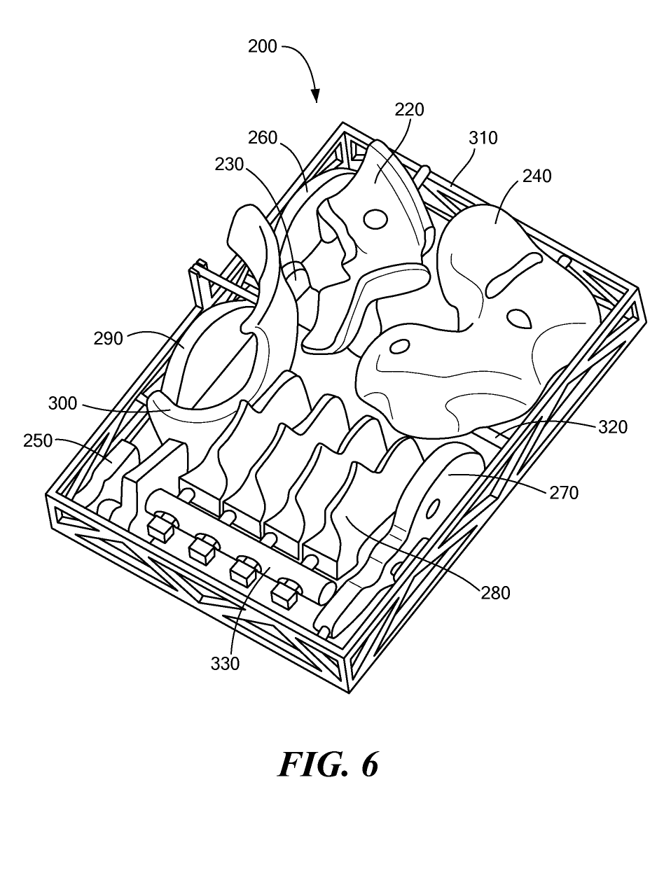

Referring to FIGS. 5 and 6, another embodiment is shown. A set of instruments is manufactured as an integrated unit 200. Unit 200 includes the patient-specific instruments to be used in a knee resurfacing operation in which a ConforMIS iDuo unicompartmental knee resurfacing implant is to be implanted in a patient. The unit 200 includes a femoral guide 220, an “L”-guide 230, a femoral gauge 240, a tibial guide 250, a 7 mm tibial sample insert 260, a tibial template 270, a set of four balancing chips 280 of graduated sizes, a 9 mm tibial sample insert 290, and a femoral trial implant 300. Each of the components is included within a frame 310, and all of the components are manufactured as a single integrated piece. The components are connected by a set of pegs 320 and connectors 330 that attaches each component to the frame 310.

In comparing unit 10 to unit 200, the individual components of unit 200 have been arranged differently to accommodate both the larger instrumentation associated with a bicompartmental implant as well as the larger components associated with a larger patient. Thus, these embodiments provide flexibility in design of instrumentation for different sizes and types of implants by allowing flexibility in the arrangement of components within the frame. The unit can be arranged for each patient, or the process can be based on a set of rules. For example, a library of arrangements can be created and used if the instrumentation set meets particular specifications associated with that arrangement. Such an embodiment allows for efficient placement of components without requiring additional design time to arrange an individual unit.

In other embodiments, the components of the integrated unit may be arranged in an order in which they are used in surgery to facilitate their use in surgery. In other embodiments, the individual components can be marked for identification. Similarly, a tab or similar structure can be incorporated near the components and include an identifying mark. In other embodiments, the unit and/or some or all of the individual components each include an identifying mark, such as a serial number, associated with a patient. In another embodiment, the components are attached to a central support member rather than a surrounding frame, other support structures or combinations of structures can be used. In another embodiment, certain components, such as related instruments, can be connected to each other instead of or in addition to being attached to the frame. In other embodiments, a tray can be included. In other embodiments, the primary assembly can be either horizontal or vertical. In other embodiments, units can be designed and/or manufactured either manually, automatically or semi-automatically. In other embodiments, various types of connections can be used, e.g., snapping or clipping members can be used to secure some or all of the components. In other embodiments, reference markers can be included on the components or the frame or other parts of the unit to allow for further processing, e.g., optical, mechanical, and electrical markers. In other embodiments, an inspection jig can be included to inspect the implant prior to surgery to make sure it meets the required specifications. In other embodiments, identifying objects, such as radio frequency identification (RFID) tags can be included.

Although implants are preferably made of metal at the present time and typically would be included separately from the instrumentation, other embodiments would allow the implant to be included in a unit with some or all of the instrumentation, including embodiments in which the implant and the instrumentation and other components are manufactured from the same materials or from different materials. Some such embodiments are an implant kit system. In some such embodiments, the kit may include an implant and a patient specific, disposable jig system, where the shape of at least a portion of an articular surface in a patient is derived and wherein both the implant and the instrumentation include at least one surface that is, at least in part, substantially a negative of the derived articular surface. In such a system, the pattern of the implant can be generated using a rapid prototyping process for subsequent casting of the implant. Similarly, the instrumentation, e.g., a patient-specific jig or other instrumentation, can be generated using a rapid prototyping system. The instrumentation can include at least one guide for adapting the joint for the implant placement. The position of the guide and the resultant adaptations of the joint surface can be used to determine at least one of the position or orientation of the implant.

In other embodiments, methods of producing an implant kit system can include various features. For example, an implant kit system can includes an implant and a patient specific, disposable jig system. The shape of at least a portion of an articular surface in a patient can be derived and one or more dimensions of the articular surface can be determined. In some embodiments, a pre-existing implant system with a desired fit can be selected. The pre-existing implant system can be adapted to the articular surface using a process, such as, without limitation, mechanical abrasion. The disposable jig or other instrumentation can include at least one surface that is, at least in part, substantially a negative of the derived articular surface. One or more components of the instrumentation can be generated using a rapid prototyping system, and it can include at least one guide for adapting the joint for the implant placement. The position of the guide and the resultant adaptations of the joint surface can be used to determine at least one of the position or orientation of the implant.

Various embodiments of the invention can be adapted and applied to implants and other devices associated with any anatomical joint including, without limitation, a spine, spinal articulations, an intervertebral disk, a facet joint, a shoulder joint, an elbow, a wrist, a hand, a finger joint, a hip, a knee, an ankle, a foot and toes. Furthermore, various embodiments can be adapted and applied to implants, instrumentation used during surgical or other procedures, and methods of using various patient-specific implants.

The embodiments described above are intended to be merely exemplary; many other embodiments including various combinations of the elements described above or other additional elements and/or additional embodiments are possible. All such variations and modifications are intended to be within the scope of various embodiments of the invention. It is intended that the scope of the invention be defined by the following claims and equivalents thereof.

What is claimed is:

1. A set of surgical instrumentation for use in a selected surgical procedure, the set comprising
a frame;
at least one individual instrument component; and
connecting means for releasably connecting said at least one individual instrument component to said frame, wherein said frame, said at least one individual instrument component and said connecting means form an integrated unit.

2. The set of surgical instrumentation of claim 1 wherein said frame and said individual instrument components are manufactured as one integrated unit.

3. The set of surgical instrumentation of claim 1 wherein the set of surgical instrumentation is disposable.

4. The set of surgical instrumentation of claim 1 wherein the selected surgical procedure is orthopedic arthroplasty.

5. The set of surgical instruments of claim 4 wherein the orthopedic arthroplasty is a knee replacement.

6. The set of surgical instruments of claim 4 wherein the orthopedic arthroplasty is a hip replacement.

7. The set of surgical instruments of claim 4 wherein the orthopedic arthroplasty is a shoulder replacement.

8. The set of surgical instruments of claim 4 wherein the orthopedic arthroplasty is an ankle replacement.

9. The set of surgical instruments of claim 1 wherein the selected surgical procedure is to be conducted on the spine, a facet joint or an intevertebral disc.

10. The set of surgical instruments of claim 1 wherein the selected surgical procedure is to be conducted on an elbow, a wrist, a hand or a finger.

11. The set of surgical instruments of claim 1 wherein the selected surgical procedure is to be conducted on a foot or a toe.

12. The set of surgical instruments of claim 1 wherein the at least one individual instrument component is patient-specific.

13. The set of surgical instruments of claim 12, further comprising at least one standard individual instrument component.

14. A set of surgical instrumentation for use in a selected surgical procedure, the set comprising
a support structure;
at least one individual instrument component; and
connecting means for releasably connecting said at least one individual instrument component to said support structure, wherein said support structure, said at least one individual instrument component and said connecting means form an integrated unit.

15. The set of surgical instrumentation of claim 14 wherein said frame and said individual instrument components are manufactured as one integrated unit.

16. The set of surgical instrumentation of claim 14 wherein the at least one individual instrument component is disposable.

17. The set of surgical instrumentation of claim 14 wherein the selected surgical procedure is orthopedic arthroplasty.

18. The set of surgical instrumentation of claim 17 wherein the orthopedic arthroplasty is a knee replacement.

19. The set of surgical instrumentation of claim 17 wherein the orthopedic arthroplasty is a hip replacement.

20. The set of surgical instrumentation of claim 17 wherein the orthopedic arthroplasty is a shoulder replacement.

21. The set of surgical instrumentation of claim 17 wherein the orthopedic arthroplasty is an ankle replacement.

22. The set of surgical instrumentation of claim 14 wherein the selected surgical procedure is to be conducted on the spine, a facet joint or an intevertebral disc.

23. The set of surgical instrumentation of claim 14 wherein the selected surgical procedure is to be conducted on an elbow, a wrist, a hand or a finger.

24. The set of surgical instrumentation of claim 14 wherein the selected surgical procedure is to be conducted on a foot or a toe.

25. The set of surgical instrumentation of claim 14 wherein the at least one individual instrument component is patient-specific.

26. The set of surgical instrumentation of claim 25, further comprising at least one standard individual instrument component.

27. A method for fabricating a set of integrated surgical instrumentation, the method comprising:
receiving a data file containing specifications of a surgical component;
designing at least one surgical instrument based on the specifications in said data file;
creating an assembly file representing the placement of at least one surgical instrument within a frame;
converting said assembly file into an STL file; and
transferring said STL file to a rapid prototyping instrument for fabrication of said set of integrated surgical instrumentation.

28. The method of claim 27 wherein the set of surgical instrumentation is configured for orthopedic surgery.

29. The method of claim 28 wherein the orthopedic surgery is joint replacement surgery.

30. The method of claim 29 wherein the joint replacement surgery is a knee replacement.

31. The method of claim 29 wherein the joint replacement surgery is a hip replacement.

32. The method of claim 29 wherein the joint replacement surgery is a shoulder replacement.

33. The method of claim 29 wherein the joint replacement surgery is an ankle replacement.

34. The method of claim 28 wherein the orthopedic surgery is to be conducted on the spine, a facet joint or an intevertebral disc.

35. The method of claim 28 wherein the orthopedic surgery is to be conducted on an elbow, a wrist, a hand or a finger.

36. The method of claim 28 wherein the orthopedic surgery is to be conducted on a foot or a toe.

37. The method of claim 27 wherein the set of surgical instrumentation is patient-specific.

38. The method of claim 27 wherein the rapid prototyping instrument performs selective laser sintering.

39. The method of claim 27 wherein the set of surgical instrumentation is disposable.

40. The method of claim 27 wherein the set of surgical instrumentation is patient-specific.

41. The method of claim 40, further comprising at least one standard individual instrument component.

42. The method of claim 27 wherein multiple sets of integrated surgical instrumentation are fabricated at the same time.

43. A method for fabricating a set of integrated surgical instrumentation, the method comprising:
receiving a data file containing specifications of a surgical component;
designing at least one surgical instrument based on the specifications in said data file;
creating an assembly file representing the connection of at least one surgical instrument to a support structure;
converting said assembly file into an STL file; and
transferring said STL file to a rapid prototyping instrument for fabrication of said set of integrated surgical instrumentation.

44. The method of claim 43 wherein multiple sets of integrated surgical instrumentation are fabricated at the same time.

45. A method for fabricating patient-specific surgical instrumentation, the method comprising:
identifying a body part for surgery;
obtaining image data of the body part to receive surgery;
deriving an electronic model of the articular surface of the surgical site;
generating a computer aided design file that includes substantially a negative of the derived articular surface;
designing at least one surgical instrument based on the specifications in said data file;
creating an assembly file representing the connection of at least one surgical instrument to a support structure;
converting said assembly file into an STL file; and
transferring said STL file to a rapid prototyping instrument for fabrication of said set of integrated surgical instrumentation.
